(19) United States
(12) Patent Application Publication
(10) Pub. No.: US 2024/0209298 A1
(43) Pub. Date: Jun. 27, 2024

(54) Device, System and Method of In-Site 4D Bioprinting Organ

(71) Applicant: Zhongtang Wang, Xiamen (CN)

(72) Inventor: Zhongtang Wang, Xiamen (CN)

(21) Appl. No.: 18/393,988

(22) Filed: Dec. 22, 2023

(30) Foreign Application Priority Data
Dec. 26, 2022 (CN) 202211671375.1

(51) Int. Cl. C12M 3/00; A61L 27/36; A61L 27/56

(52) U.S. Cl. CPC C12M 21/08; A61L 27/3637; A61L 27/56; A61L 2400/06; A61L 2430/00

(57) ABSTRACT

A device of in-situ 4D bioprinting organ includes an implantable component, a connectable component and an external component. The implantable component includes a scaffold for shaping an organ defect, a microfluidic system for printing bio ink, and an intelligent monitoring unit. The external component includes a filling and draining unit, a power, a control unit for providing support to the implantable component. The implantable component can be placed in a body or installed on a surface of the body and connected to the organ defect. The bio ink can be accurately layered and segmented according to an algorithm onto a wound surface of the organ defect, thereby implementing the in-situ bioprinting organ.

### CROSS REFERENCE OF RELATED APPLICATION

This application is a non-provisional application that claims the benefit of priority under 35U.S.C. § 119(e) to a Chinese application, application number 202211671375.1, filed Dec. 26, 2022, which is incorporated herewith by reference in its entirety.

### BACKGROUND OF THE PRESENT INVENTION

### Field of Invention

The present invention relates to a field of medical device, and more particularly to a device, system and method of in-situ four-dimensional 4D bioprinting organ.

### Description of Related Arts

Organ transplantation is an effective alternative medical measurement for a patient with a disabled organ, but due to a shortage of donors, the vast majority of patients might not have the opportunity to be treated timely. In addition, when part of organ is destroyed because of trauma or tumor resection, the residual organ might not sufficient enough to meet a functional need. Therefore the organ transplantation still needs to be considered.

Three-dimensional (3D) bioprinting organ is one of the choices to solve the shortage of donors. Due to some unresolved technologies such as blood vessel construction, secretion and excretion pipelines, and organ function realization after transplantation, a huge clinical transformation gap is needed to overcome. In-situ 3D bioprinting of wounds can be used to repair skin defects, which utilizes the human body as a natural bioreactor, and has a potential advantage in narrowing the clinical transformation gap (Albanna et al. Sci Rep. 2019 Feb. 12; 9 (1): 1856).

The prior art of 3D bioprinting organ has limitations, mainly including: (1) the transplantation of tissues and/or organs printed in vitro into the human body does not have organ functions, and even the transplanted tissues and/or organs cannot survive normally; (2) in vitro printing of tissues and/or organs requires a preparation of a large number of seed cells, which might take a long term to prepare before transplantation; (3) in vivo printing of tissue and/or organs can be used for repairing skin defects only, and cannot yet repair complex surface organ defects such as ears and nose, even more unable to repair deep organ defects in the body; (4) because 3D printed tissues and/or organs in vitro or in vivo lack a good survival microenvironment, it is difficult to obtain a well healing result.

After part of the organs is removed due to trauma or tumor, a tissue regeneration microenvironment is constructed in-situ at a residual end of the residual organs with a help of 3D bioprinting technology, and printed in layers and stages as needed, so that the residual organs can be induced to regenerate into complete organs so as to achieve a well health. Similarly, a donor organ can be divided into a plurality of relatively independent grafts, which can be transplanted to a plurality of patients. With the help of 3D bioprinting technology, the tissue regeneration microenvironment can be constructed at the residual end of each graft and printed in layers and stages as required, which will induce each graft to regenerate into a complete organ, thus allowing more patients to obtain the opportunity of organ transplantation.

### SUMMARY OF THE PRESENT INVENTION

A portable in vivo in-situ 4D bioprinting device is provided, which can be placed into a body or installed on the body surface connected with an organ residual to build a tissue regeneration microenvironment, and a bio ink can be inkjet to a wound surface of the organ residual, in layers and stages, so as to induce the tissue regeneration of the organ residual to be a complete organ.

On the one hand, an embodiment of the present invention provides a device of in-situ 4D bioprinting organ, which may comprise an implantable component, a connectable component and an external component. The implantable component can be configured to be placed in a body or installed on the body surface and connect a defective organ, inkjet a bio ink and/or artificial amniotic fluid and induce tissue regeneration of the defective organ. The external component can be configured to provide a support for the implantable component, and the connectable component can be configured to connect the implantable component and the external component.

Further, the implantable component may comprise a scaffold for shaping an organ defect, a microfluidic system, and an intelligent monitoring unit. The scaffold for shaping an organ defect at least includes an inner wall, an outer shell and a plurality of first micropores configured to shape a structure of the organ defect. The microfluidic system may comprise a capillary net, a plurality of second micropores and a first connective pipe configured to inkjet the bio ink and/or artificial amniotic fluid. The intelligent monitoring unit may comprise a plurality of sensors, which comprises at least one or more combinations of a visual sensor, a near-infrared sensor, a pH sensor, a temperature sensor, a microbial sensor and a biochemical sensor configured to collect data of the organ defect area.

Preferably, the scaffold for shaping an organ defect can be customized based on a shape and a size of the organ defect, usually made of biodegradable materials. Preferably, a 3D printing technology can be selected for processing and production of the scaffold for shaping an organ defect, and the 3D modeling can refer to an imaging data of the organ defect and/or a healthy side of the organ.

Further, the inner wall can be a medullary cavity of the organ to be repaired in a defect area, such as a cerebral ventricle, renal pelvis and calyces, uterine cavity, atrium or ventricle of a heart, and tracheobronchial of a lung. Alternately, organs without medullary cavity structure can simplify or omit the inner wall, such as a spleen, thyroid, adrenal gland, ovary, and liver.

Further, the outer shell can be an outer wall of the organ to be repaired in a defect area, and preferably, the outer shell has an interface for connecting the connectable component.

Preferably, the capillary net can be arranged in a chamber between the inner wall and the outer shell, which matches the shape and size of the chamber and can slide freely along the inner wall and outer shell. The chamber can be configured to accommodate the organ to be repaired in a defect area.

Preferably, the first micropore can be arranged on the inner wall with a pore size no more than 2 u m. The first micropore can block cells but allow liquid to pass through. Preferably, the first micropore can be a semi permeable membrane that selectively allows some liquid components to pass through.

Preferably, the first micropore can be arranged on the outer shell with a pore size no more than 5 u m. The first micropore can block cells but allow liquid to pass through. Preferably, the first micropore can be a semi permeable membrane that selectively allows some liquid components to pass through.

Preferably, the second micropore can be arranged on a side of the capillary net towards a residual end of the organ configured to spray the bio ink and/or artificial amniotic fluid, with a pore size from 50 μm to 2000 μm. More preferably, the second micropore can be with a pore size from 200 μm-800 μm.

Further, the scaffold for shaping an organ defect may comprise a plurality of paired inner walls and outer shells configured to shape a plurality of structures of the organ defect area, such as a left and right brain, lungs, and heart.

Further, the microfluidic system may comprise a plurality of microfluidic subsystem, which can be arranged in a plurality of chambers formed between paired inner walls and outer shells configured to inkjet the bio ink to achieve a plurality of structures in organ defect areas and form a plurality of lobes or partitions or multiple organs.

Further, the implantable component may comprise a temperature control unit, which can include a heating element for maintaining a temperature from 37.5° C. to 39.5° C. in the chamber.

Further, the external component may comprise a filling unit, a drainage unit, and a control unit. The filling unit can include a second connecting tube, a first injectable pot, a liquid storage bag, a first micropump and a micro valve configured to inject the bio ink and/or artificial amniotic fluid into the microfluidic system. The drainage unit can include a third connecting tube, a second micropump, a waste liquid bag and a second injectable pot configured to discharge waste liquid from an organ defect area. The control unit can be configured to control the first micropump, the second micropump, and the micro valve.

Further, the control unit may comprise at least a data storage processor and a wireless communication subunit.

Further, the filling unit can include a plurality of second connecting tubes, a plurality of first injectable pots, a plurality of liquid storage bags, a plurality of first micropumps, and a plurality of micro valves configured to inkjet segmented bio ink and/or artificial amniotic fluid of different formulations into a plurality of microfluidic subsystems.

Further, the implantable component can be used on a surface of an organ defect, such as a skin defect, breast defect, nose defect, lip defect, auricle defect, eyeball defect, external genitalia defect, limb defect, trunk defect, or in a natural lacunar organ defect, such as a tongue defect, anal defect, intestinal wall defect, vaginal defect, uterine defect, tracheal defect, configured to spray the bio ink to induce tissue regeneration of the organ defect.

Preferably, the implantable component can create a simulated amniotic cavity environment locally in the organ defect area. The scaffold for the organ defect can be made of degradable or non-degradable transparent materials.

Further, the bio ink may comprise at least one or more combinations of seed cells, collagen, cellulose, hyaluronic acid, and growth factors. The seed cells can include stem cells or pluripotent stem cells, mature organ cells, endothelial cells, and epithelial cells.

Further, the artificial amniotic fluid can be configured to fill the chamber, so that the chamber has a microenvironment similar to the amniotic cavity and promotes a regeneration of organ defect. Preferably, the artificial amniotic fluid may comprise additives, which can include one or more combinations of cytokines, growth factors, hormones, hemostatic agents, antibiotics, and enzymes.

Further, the external component may comprise a micro motor, preferably a micro stepper motor, which can be connected to a capillary net configured to drive the capillary net to move back and forth along the inner wall and outer shell.

Further, the external component may comprise a power, which includes a rechargeable battery for providing electricity.

Further, the connectable component may comprise a base configured to connect the internal and external parts of the device and anchor the implantable component and the external component. The base may comprise an abutment and a through hole. The abutment can be fixed to a surface of a body. The through hole can configured to penetrate power transmission units, connect tubes, circuits, and communication lines. Preferably, the through hole includes a one-way valve configured to prevent external air from entering the chamber and liquid from overflowing outside the chamber.

Further, the abutment can be configured to install a micro motor, the filling unit, the drainage unit, and the control unit.

Further, the connectable component may comprise a connector, which can be detachably connected to an interface of the implantable component.

Further, the connectable component may comprise a guide tube, one end of which is connected to the through hole, and the other end is detachably connected to the interface of the implantable component through a joint.

On the other hand, an embodiment of the present invention provides a system for in-situ 4D bioprinting organ, which may comprise a device of in-situ 4D bioprinting organ, a bio ink, an artificial amniotic fluid, a IoT operating system, an application program, and an algorithm.

Further, the application program may comprise a mobile program and a server program.

Further, the algorithm may comprise a monitoring data fusion analysis, a bio ink spraying scheme, an artificial amniotic fluid filling and drainage scheme, and a recognition of wound healing process.

Further, the system may comprise a 5G, a cloud server, and an intelligent terminal.

Further, the system may comprise a medical care unit.

On the other hand, an embodiment of the present invention provides a method for in-situ 4D bioprinting organ, including steps of:
S1: customizing a scaffold for shaping an organ defect and a microfluidic system, a scaffold for shaping an organ defect including an inner wall, an outer shell, and a plurality of first micropores, and a microfluidic system including a capillary net, a plurality of second micropores, and a first connecting tube;
S2: preparing a bio ink and/or artificial amniotic fluid, the bio ink including seed cells, extracellular matrix, and growth factors;
S3: assembling the scaffold for shaping an organ defect, a microfluidic system and a plurality of sensors, which being folded or compressed into a body through a minimally invasive incision or installed on a body surface, and connected and bonded with a residual of organ defect;
S4: installing a connectable component and an external component; and
S5: spraying the bio ink and filling the artificial amniotic fluid according to a procedure, and providing a local tissue regeneration microenvironment, thus inducing a tissue regeneration.

Further, the method for in-situ 4D bioprinting organ can include real-time collecting local data of the organ defect area by the sensors, which includes a wound microcirculation data, a tissue regeneration progress data, a temperature data, and a pH data.

Further, the method for in-situ 4D bioprinting organ can include completing an organ regeneration, removing the connectable component and external component, removing the microfluidic system and sensors, and degrading or removing the scaffold for shaping an organ defect.

Further, the method for in-situ 4D bioprinting organ can include printing and reconstructing an outer auricle in an external ear defect area.

Further, the method for in-situ 4D bioprinting organ can include printing and reconstructing a nose in a nasal defect area.

Further, the method for in-situ 4D bioprinting organ can include printing and reconstructing a breast in a breast defect area.

Further, the method for in-situ 4D bioprinting organ can include printing and reconstructing a liver in a liver defect area.

Further, the method for in-situ 4D bioprinting organ can include printing and reconstructing a kidney in a kidney defect area.

Further, the method for in-situ 4D bioprinting organ can include printing and reconstructing a brain in a brain defect area.

Further, the method for in-situ 4D bioprinting organ can include printing and reconstructing a trunk limb in a trunk limb defect area.

Further, the method for in-situ 4D bioprinting organ can include printing and reconstructing a heart in a heart defect area.

Further, the method for in-situ 4D bioprinting organ can include printing and reconstructing a genitalia in a genital defect area.

Further, the method for in-situ 4D bioprinting organ can include printing and reconstructing a tongue in a tongue defect area.

Further, the method for in-situ 4D bioprinting organ can include printing and reconstructing a lung in a lung defect area.

Further, the method for in-situ 4D bioprinting organ can include printing and reconstructing a uterine in a uterine defect area.

Further, the method for in-situ 4D bioprinting organ can include printing and reconstructing a face in a facial defect area.

Further, the method for in-situ 4D bioprinting organ can include printing and reconstructing a scalp in a scalp defect area.

Further, the method for in-situ 4D bioprinting organ can include printing and reconstructing a scalp and skull in a scalp and skull defect area.

Further, the method for in-situ 4D bioprinting organ can include printing and reconstructing fingers in finger defect areas.

Further, the method for in-situ 4D bioprinting organ can include printing and reconstructing a toe in a toe defect area.

Further, the method for in-situ 4D bioprinting organ can include printing and

reconstructing an anus in a anal defect area.

Further, the method for in-situ 4D bioprinting organ can include printing and reconstructing a gastrointestinal tract in a gastrointestinal defect area.

Further, the method for in-situ 4D bioprinting organ can include printing and reconstructing a pancreas in a pancreatic defect area.

Further, the method for in-situ 4D bioprinting organ can include printing and reconstructing a spleen in a spleen defect area.

### Beneficial Effects

1. The device of in-situ 4D bioprinting organ has a compact and well-designed structure, which is easy to carry. The implantable component can be inserted into a human body through a minimally invasive incision or installed on a surface of the human body and bonded to an organ defect. The wound surface of the organ defect can be used as a bioreactor to repair deep organ or surface organ defects in the human body, thereby simplifying the process of inducing seed cells to differentiate into organs, making the in-situ bioprinting organs have organ functions.

2. When implementing bioprinting with the device of in-situ 4D bioprinting organ, a bio ink can be sprayed in layers and stages as needed until the organ defect completes regeneration. On the one hand, a small amounts of seed cells are required each time, reducing a waiting time for preparing the bio ink. On the other hand, a thickness of the seed cell layer is thin, and blood vessels of the wound surface of the organ defect can grow into the seed cell layer in a short period of time, ensuring that the seed cells receive sufficient nutrition and survive normally. The microenvironment of the organ defect can induce a new tissue to have organ function according to a self-organization and self-assembly law of nature, so as to avoid a difficulty in survival of the organ printed out at a time of transplantation and the inability to connect the blood vessels with the secretion and excretion pipelines, which will not have organ function.

3. When implementing bioprinting with the device of in-situ 4D bioprinting organ, the bio ink can be sprayed in layers onto a freshly prepared wound at the residual end of the organ defect. The thickness of each layer of the bio ink can be 1 mm or several millimeters. After the bio ink completely forms organ tissue in this layer, the bio ink can be sprayed again onto the residual wound of the organ defect. The time interval between the two sprays can be intelligently or manually determined.

4. When the device of in-situ 4D bioprinting organ implements bioprinting and layered spray printing, the bio ink formula can be flexibly formulated according to a need of the organ defect, so that the new organ tissue has relatively complete functions and structures, such as the solid cells and outer wall cells of the organs, a cartilage, subcutaneous fat and skin of the outer ear.

5. The device of in-situ 4D bioprinting organ can use a microfluidic system to spray the bio ink. Due to a small dead space volume of the microfluidic system, a waste of the bio ink can be avoided.

6. The implantable component of the device of in-situ 4D bioprinting organ can be placed on a surface of the human body to repair complex surface organ defects such as an eye, an ear, a nose, a breast, a limb, a trunk, and/or external genitalia.

7. The implantable component of the device of in-situ 4D bioprinting organ can be placed in a natural lumen to repair complex organ defects such as a tongue, larynx, trachea, uterus, and intestines.

8. The device of in-situ 4D bioprinting organ can fill a chamber of a printing area with artificial amniotic fluid, and can add growth factors, cytokines, antibiotics, hormones, etc. to the artificial amniotic fluid according to local needs, so as to provide a suitable healing microenvironment for bioprinting tissues and organs and promote the regeneration of the organs. In addition, analyzing a composition of a drainage fluid can be used to understand a change in secretion and metabolism of the regenerative tissue.

9. The device of in-situ 4D bioprinting organ can be equipped with a variety of sensors, intelligent control units and wireless communication units. A medical expert can monitor the local microenvironment data of bioprinting in real time through intelligent terminals, and implement remote control, so that a patient does not need to be hospitalized.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

In order to provide a clearer explanation of the technical solution of the embodiments of the present invention, a brief introduction will be given to a plurality of accompanying drawings required in the description of the embodiments. It is evident that the accompanying drawings in the following description are only some embodiments of the present invention. For those skilled in the art, other accompanying drawings can be obtained based on these drawings without any creative effort.

In addition, the accompanying drawings are only schematic illustrations of the present invention and are not necessarily drawn to scale. The same reference numerals in the figure represent the same or similar parts, so repeated descriptions of them will be omitted. Some of the block diagrams shown in the accompanying drawings are functional entities that do not necessarily correspond to physically or logically independent entities, and can be implemented in one or more hardware modules or component combinations or software.

FIG. 1 is a schematic view of a functional module structure of a device of in-situ 4D bioprinting organ according to an embodiment of the present invention.

FIG. 2 is a profile schematic view of a cross-sectional structure of a device of in-situ 4D bioprinting organ according to an embodiment of the present invention.

FIG. 3 is another profile schematic view of a cross-sectional structure of a device of in-situ 4D bioprinting organ according to an embodiment of the present invention.

FIG. 4 is a profile schematic view of a cross-sectional structure of an implantable component of a device of in-situ 4D bioprinting organ according to an embodiment of the present invention.

FIG. 5A is a schematic inferior view of the cross-sectional structure of a device of in-situ 4D bioprinting organ according to an embodiment of the present invention.

FIG. 5B is a schematic vertical view of the cross-sectional structure of a device of in-situ 4D bioprinting organ according to an embodiment of the present invention.

FIG. 6A is a schematic inferior view of a capillary net structure of an outer shell of a device of in-situ 4D bioprinting organ according to an embodiment of the present invention.

FIG. 6B is a schematic vertical view of a capillary net structure of an outer shell of a device of in-situ 4D bioprinting organ according to an embodiment of the present invention.

FIG. 7A is a schematic inferior view of a capillary net structure of an inner wall of a device of in-situ 4D bioprinting organ according to an embodiment of the present invention.

FIG. 7B is a schematic vertical view of a capillary net structure of an inner wall of a device of in-situ 4D bioprinting organ according to an embodiment of the present invention.

FIG. 8A is a schematic inferior view of a capillary net structure of a main body of a device of in-situ 4D bioprinting organ according to an embodiment of the present invention.

FIG. 8B is a schematic vertical view of a capillary net structure of a main body of a device of in-situ 4D bioprinting organ according to an embodiment of the present invention.

FIG. 9A is a profile schematic view illustrating a cross-sectional structure of a connectable component of a device of in-situ 4D bioprinting organ according to an embodiment of the present invention.

FIG. 9B is a second profile schematic view illustrating a cross-sectional structure of a connectable component of a device of in-situ 4D bioprinting organ according to an embodiment of the present invention.

FIG. 9C is a third profile schematic view illustrating a cross-sectional structure of a connectable component of a device of in-situ 4D bioprinting organ according to an embodiment of the present invention.

FIG. 9D is a fourth profile schematic view illustrating a cross-sectional structure of a connectable component of a device of in-situ 4D bioprinting organ according to an embodiment of the present invention.

FIG. 10A is a schematic view illustrating the structure of a filling unit of a device of in-situ 4D bioprinting organ according to an embodiment of the present invention.

FIG. 10B is a second schematic view illustrating the structure of a filling unit of a device of in-situ 4D bioprinting organ according to an embodiment of the present invention.

FIG. 10C is a third schematic view illustrating the structure of a filling unit of a device of in-situ 4D bioprinting organ according to an embodiment of the present invention.

FIG. 11 is a schematic view illustrating a drainage unit structure of a device of in-situ 4D bioprinting organ according to an embodiment of the present invention.

FIG. 12 is a schematic flow of a method for an in-situ 4D bioprinting organ according to an embodiment of the present invention.

### DETAILED DESCRIPTION OF THE PREFERRED EMBODIMENT

In order to make the objects, technical solutions, and advantages of the present invention clearer, a further detailed explanation of the present invention in conjunction with embodiments as follow will be provided. It should be understood that the specific embodiments described here are only used to explain the present invention and are not intended to limit it.

It should be noted that the above, below, left, right, far, near, front, back, front, back, and other directional words in the embodiments are only relative concepts or refer to a normal use status of the product, and should not be considered restrictive.

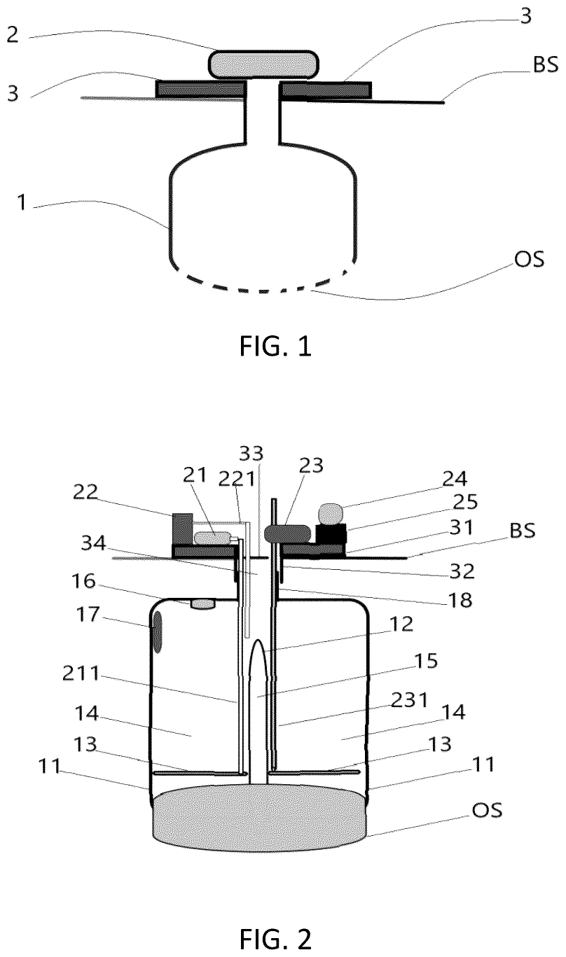

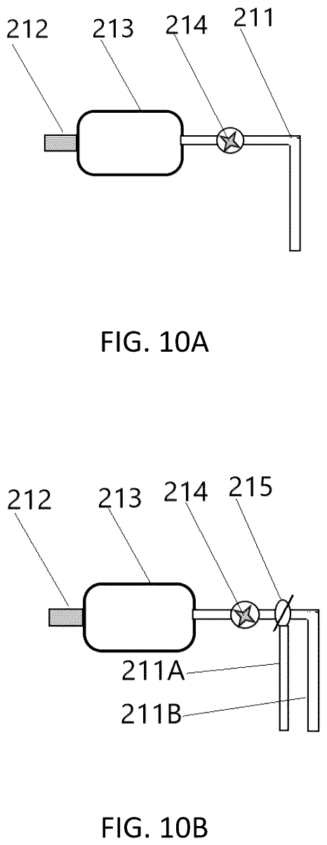

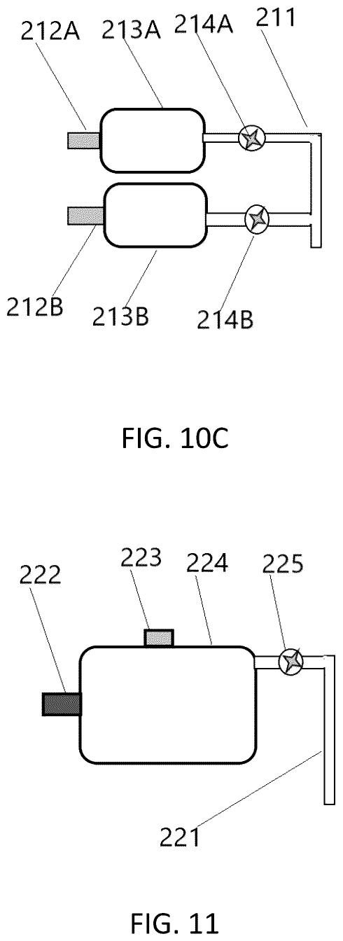

Referring to FIG. 1 to FIG. 12, a device of in-situ 4D (four-dimensional) bioprinting organ according to a preferred embodiment of the present invention can be configured to insert into a human body or install on the human body surface and connect to an organ defect, spray a bio ink and/or artificial amniotic fluid, induce a regeneration and reconstruction of the organ defect, and repair a partial organ defect or reconstruct a complete organ.

As shown in FIG. 1, a device of in-situ 4D bioprinting organ according to the above preferred embodiment of the present invention comprises an implantable component 1, an external component 2 and a connectable component 3. The implantable component 1 can be inserted into a human body through a surgical incision or natural lumen or installed on a human body surface (BS) and connected to an organ defect surface (OS), wherein a bio ink can be sprayed into the OS in layers and stages as needed, and an artificial amniotic fluid can be sprayed into the OS as needed, inducing a tissue regeneration in a defect area of internal organs or surface organs. The external component 2 can provide a power, a communication, a control, and a monitoring support for the implantable component 1, and the connectable component 3 can be anchored to the BS to fix and connect the implantable component 1 and the external component 2.

It should be noted that the connectable component 3 can be attached to the human body surface and can be removed or replaced if necessary. The connectable component 3 can be detachably connected to the external component 2, and the external component 2 can be removed or replaced if necessary.

As shown in FIGS. 2 and 5-8, an implantable component 1 of a device of in-situ 4D bioprinting organ according to the preferred embodiment of the present invention may comprise a scaffold for shaping an organ defect, which comprises an inner wall 12, an outer shell 11 and a plurality of first micropores 111 configured to shape a structure of the organ defect. The implantable component 1 may comprise a microfluidic system, which comprises a capillary net 13, a plurality of second micropores 132, and a first connective pipe 133 configured to inkjet the bio ink and/or artificial amniotic fluid. The implantable component 1 may comprise an intelligent monitoring unit 16, which may comprise at least one or more combinations of visual sensors, near-infrared sensors, pH sensors, temperature sensors, microbial sensors, and biochemical sensors configured to collect data of the organ defect area.

The scaffold for shaping an organ defect can be customized based on a shape and a size of the organ defect, usually made of biodegradable materials. More preferably, a 3D printing technology can be selected for processing and production of the scaffold for shaping an organ defect, and the 3D modeling can refer to an imaging data of the organ defect and/or a healthy side of the organ.

The inner wall 12 of the scaffold for shaping an organ defect can be usually a semi closed structure which can form an inner cavity 15. The inner cavity 15 may comprise at least one open end, which can be connected to a natural pore of a residual of OS in the organ defect area, such as a ventricle of a brain, a renal pelvis and calyces of a kidney, a uterine cavity of a uterus, an atrium or ventricle of a heart, a tracheobronchial organ of a lung, a nasal cavity and nostrils of a nose, configured to form a medullary cavity of a regenerative organ in the organ defect area. A shape and size of the inner wall 12 and inner cavity 15 can be consistent with a potential medullary cavity of the residual of OS in the organ defect area. In addition, the first micropores 111 can be set on the inner wall 12 with a pore size usually no more than 2 μm. The first micropores 111 can block cells but allow liquid to pass through, and preferably, the first micropore 111 is a semi permeable membrane that selectively allows some liquid components to pass through.

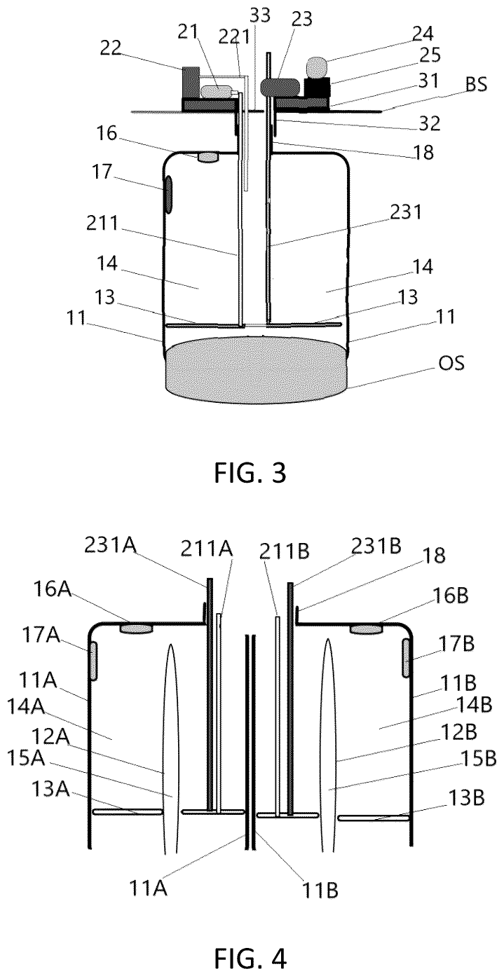

Alternatively, as shown in FIG. 3, an inner wall 12 of an in situ 4D bioprinting organ device according to the preferred embodiment of the present invention can be simplified or omitted for an organ defect area without medullary cavity structure or medullary cavity structure defect, such as a spleen, a thyroid, an adrenal gland, an ovary, a liver, a scalp skull, an outer ear, a tongue, a trunk or limbs without obvious medullary cavity structure. The inner wall 12 can be used for regeneration of damaged area with obvious medullary organ such as a brain, a kidney, a uterus, a heart, a lung, a nose, etc.

The outer shell 11 of the scaffold for shaping an organ defect can be a semi enclosed structure, which can serve as an outer wall of a regenerated organ in the organ defect area. An upper end of the outer shell 11 can be equipped with an interface 18 configured to connect the connectable component 3, and a lower end of the outer shell 11 can be sealed and bonded with the residual organ defect of the OS. The outer shell 11 can form a chamber 14 with the inner wall 12, and the chamber 14 can be used to set up a capillary net 13 configured for spraying a bio ink and artificial amniotic fluid. A shape and a size of the capillary net 13 can match the chamber 14, and can slide freely along the inner wall 12 and the outer shell 11. The chamber 14 can be used to accommodate regenerative organ. The shell 11 can be equipped with a first micropore 111 with a pore size no more than 5 μm. The first micropore 111 can block cells but allow liquid to pass through, preferably, the first micropore 111 is a semi permeable membrane that selectively allows some liquid components to pass through. In addition, the chamber 14 formed by the outer shell 11 and the inner wall 12 can provide sufficient space for the regeneration of a defective organ, avoiding the location of the defective organ being occupied by surrounding organs or tissues, which affects the regeneration of new tissues. In addition, the outer shell 11 and inner wall 12 of the scaffold for shaping an organ defect can be equipped without the first micropore 111 to prevent an overflow of the bio ink and/or artificial amniotic fluid in the chamber 14, and to prevent a foreign object such as external bacteria from entering the chamber 14.

Preferably, the chamber 14 may comprise a temperature control unit 17, which can include a heating element to maintain a temperature from 37.5° C. to 39.5° C. in the chamber 14 and provide an optimal temperature environment for the regeneration of the organ defect.

Preferably, the intelligent monitoring unit 16 may comprise a visual sensor, a near-infrared sensor, a pH sensor, and a temperature sensor, which can respectively be configured to collect a morphological data of the organ defect area of the chamber 14, a wound microcirculation data, a pH and a temperature data of the wound microenvironment to provide a decision-making basis for intelligent or human intervention. The intelligent monitoring unit 16 may comprise a plurality of microbial sensors and biochemical sensors, which can be respectively configured to collect a microbial infection data and a regenerative organ secretion and a metabolism data in the organ defect area of the chamber 14, provide a decision-making basis for intelligent or human intervention. Of course, by regularly or irregularly collecting liquid from the chamber 14 and sending it to a laboratory for testing, the microbial sensors and biochemical sensors can be eliminated. In addition, the sensors of intelligent monitoring unit can be integrated into an integrated structure and installed on an inner side of the outer shell 11 or an outer side of the inner wall 12, or can be distributed in a dispersed structure and installed on the inner side of the outer shell 11 or the outer side of the inner wall 12 or the capillary net 13.

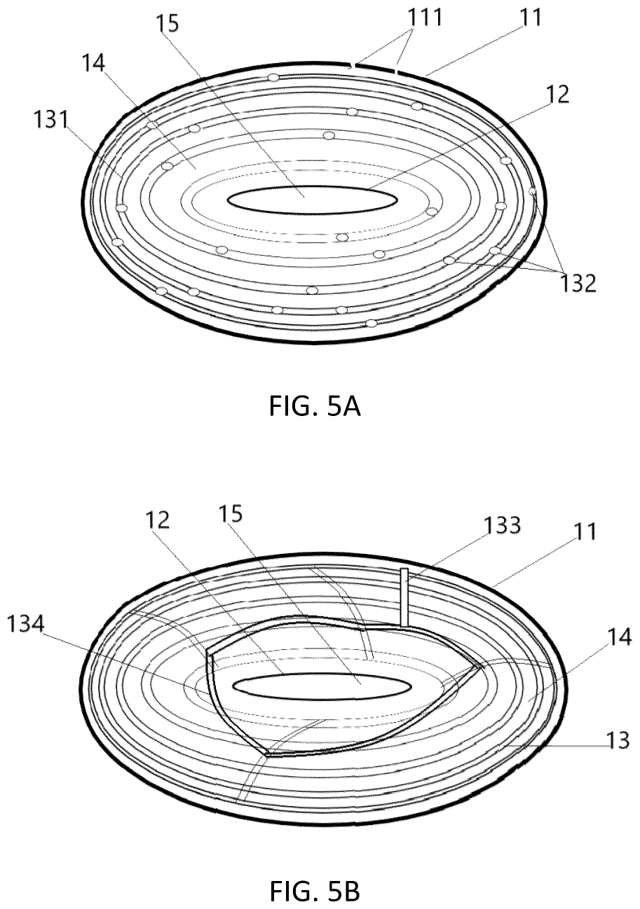

As shown in FIGS. 5A and 5B, a capillary net 13 of an in situ 4D bioprinting organ device of an embodiment of the present invention can be arranged in a circular shape around an inner wall 12 or can be arranged in a mesh or grid shape or any shape. The capillary net 13 can be made of transparent medical polymer materials, such as polyvinyl chloride (PVC). The capillary net 13 may comprise a capillary 131, a plurality of second micropores 132, a first connecting tube 133, and a plurality of traffic branch tubes 134. A diameter of the capillary 131 is usually from 100 μm to 2000 μm, preferably from 200 μm to 800 μm. The second micropores 132 can be scattered on a side of the capillary 131 towards a residual end of the organ defect. A spacing between the second micropores 132 can be from 500 μm to 5000 μm, with an aperture from 50 μm to 1000 μm, preferably from 200 μm to 500 μm. The traffic branch tubes 134 can be arranged between the capillaries 131 configured to connect the capillaries 131. One end of the first connecting tube 133 can be connected to the capillaries 131 or the traffic branch tubes 134, and the other end can connect to an external component 2.

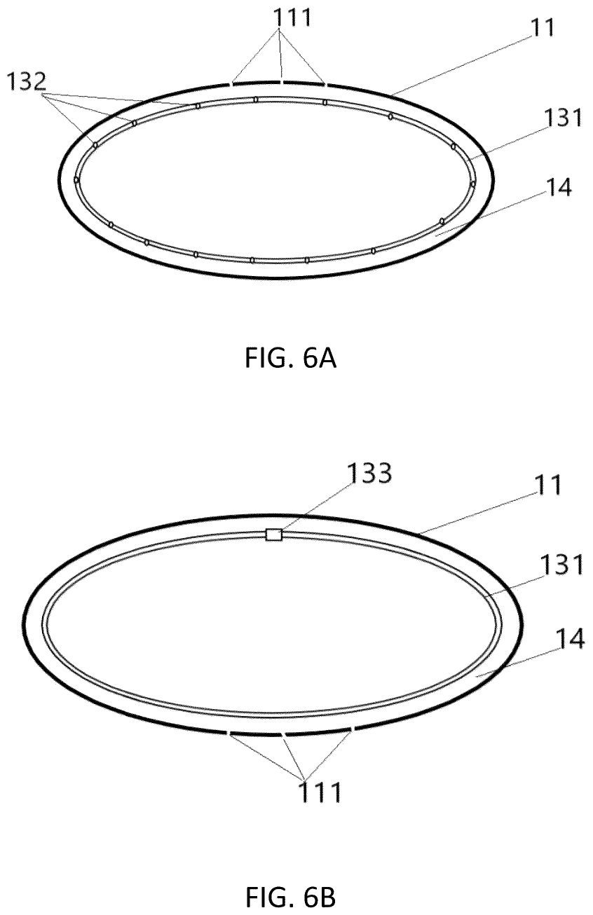

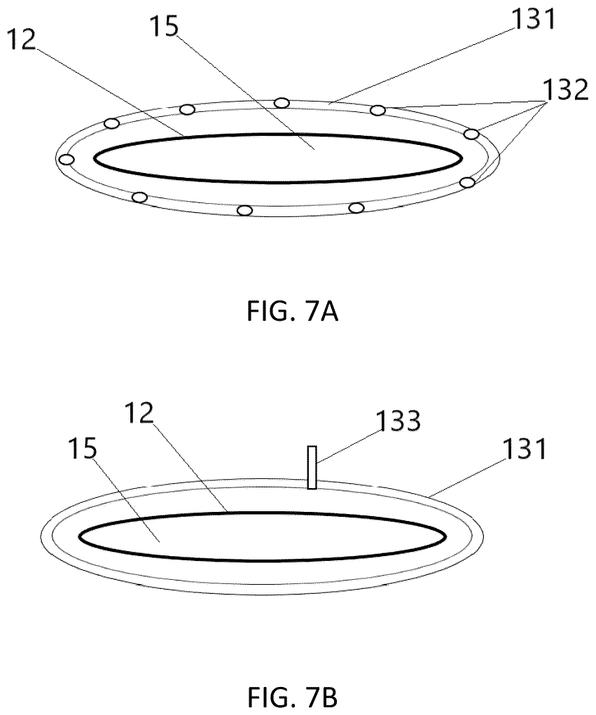

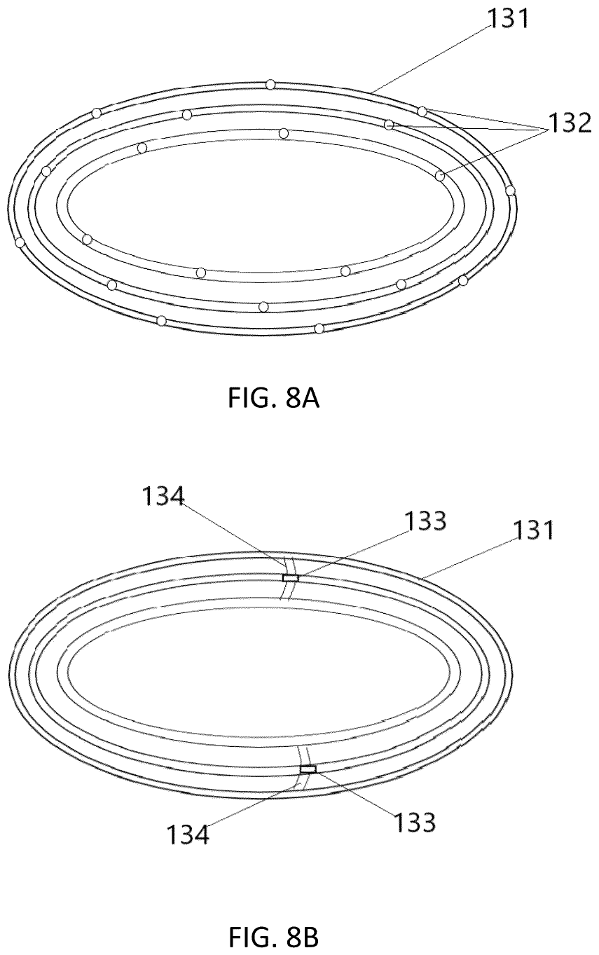

As shown in FIGS. 6A and 6B, a capillary net 13 near an outer shell 11 of an in situ 4D bioprinting organ device of an embodiment of the present invention may comprise a capillary 131, a plurality of second micropores 132, and a first connecting tube 133, configured to spray a bio ink that forms an outer membrane of a viscus or a skin and subcutaneous tissues of a human body surface organ, such as a visceral membrane layer of a liver, a skin layer of an outer ear. As shown in FIGS. 7A and 7B, a capillary net 13 near an inner wall 12 of an in situ 4D bioprinting organ device according to an embodiment of the present invention may comprise a capillary 131, a plurality of second micropores 132, and a first connecting tube 133, configured to inkjet a bio ink that forms an inner membrane of a viscus organ or a mucosal epithelium of a surface organ, such as a brain ventricular membrane, a nasal mucosa. As shown in FIGS. 8A and 8B, a capillary net 13 near an outer shell 11 of an in situ 4D bioprinting organ device of an embodiment of the present invention may comprise a capillary 131, a plurality of second micropores 132, a first connecting tube 133, and a plurality of traffic branch tubes 134, configured to inkjet a bio ink that forms a structure of a solid organ or a support structure of a surface organ, such as liver lobes of a liver, a cartilage of an outer ear. Of course, the capillary net 13 can be personalized based on a type of organ or a location of the organ defect.

As shown in FIG. 4, an implantable component 1 of a device of in-situ 4D bioprinting organ of an embodiment of the present invention may comprise an inner wall 12A, an outer shell 11A, a capillary net 13A, a chamber 14A, an inner cavity 15A, an intelligent monitoring unit 16A, a temperature control unit 17A, an inner wall 12B, an outer shell 11B, a capillary net 13B, a chamber 14B, an inner cavity 15B, an intelligent monitoring unit 16B, and a temperature control unit 17B. The inner wall 12B can enclose and form the inner cavity 15B. The chamber 14B can be formed between the inner wall 12B and the outer shell 11B, and the chamber 14B can accommodate the capillary net 13B, the intelligent monitoring unit 16B, and the temperature control unit 17B, and can be detachably and fixedly connected to a connectable component 3 through a first interface 18. It should be noted that the implantable component 1 of the device of in-situ 4D bioprinting organ shown in FIG. 4 can include two sets of relatively independent printing structures. In fact, the implantable component 1 can include three or more sets of relatively independent printing structures according to a need. A plurality of sets of inner walls 12, outer shells 11, capillary nets 13, chambers 14, inner cavities 15, intelligent monitoring units 16, and temperature control units 17 can be used to shape multiple structures of organ defect. For example, for different lobes of a left and right brain, lungs, heart, or a same organ such as liver, a plurality of capillary nets 13 can be set in a plurality of chambers 14 to spray the bio ink to achieve reconstruction of a plurality of structures, lobes, partitions, or organs in organ defect areas.

As shown in FIGS. 2, 3 and 9A-9D, a connectable component 3 of a device of in-situ 4D bioprinting organ of an embodiment of the present invention may comprise a base 31, a vertical tube 32, a sealing valve 33, a through-hole 34, and a second interface 35. The base 31 can be adhered or bound to a surface of the human body, and the vertical tube 32 passes through the base 31 and can be fixedly connected to the base 31. An outer side of the vertical tube 32 can be the sealing valve 33, an inner side of the sealing valve of the vertical tube 32 can be the through hole 34, and a lower end of the vertical tube 32 can be the second interface 35. The base 31 can be equipped with a subunit that fix the external component 2 and connect with the implantable component 1 in the human body or on a human body surface. The vertical tube 32 can be used for threading power transmission units, connecting pipelines, circuits, and communication lines. The sealing valve 33 can be preferably a one-way valve to prevent external air from entering the chamber 14 and liquid from overflowing from the human body. The second interface 35 can be used for detachable connection with the first interface 18 of the implantable component 1.

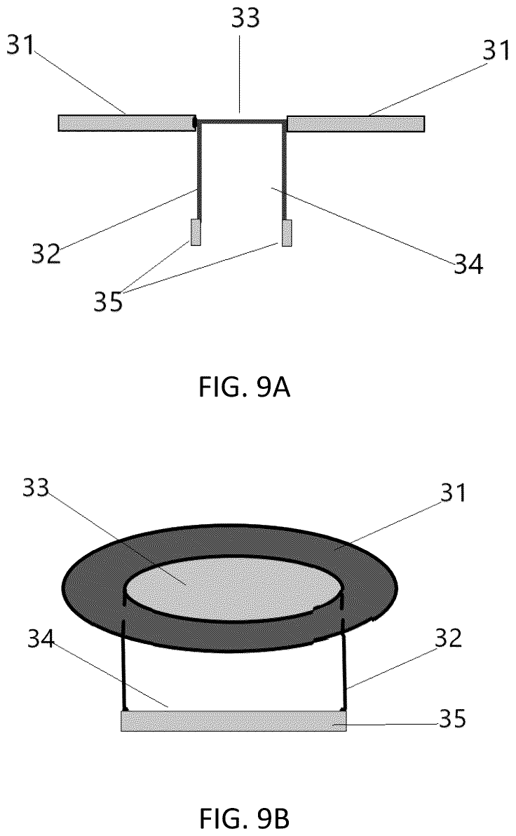

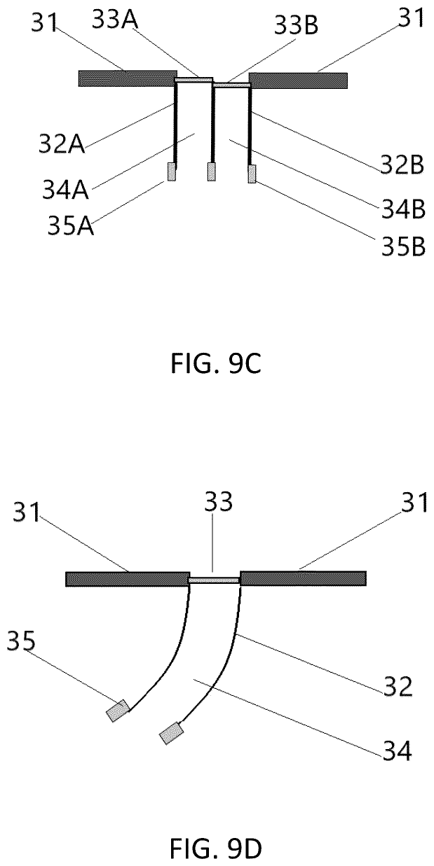

As shown in FIG. 9B, a base 31 according to the preferred embodiment of the present invention can be in a shape of a disc, a square, or any other shape. If used for bioprinting a deep organ defect, the base 31 can be bound or bonded and fixed above an incision, and the vertical tube 32 can be placed in a deep part of the human body and detachably connected to the first interface 18 of the implantable component 1 through the second interface 35. If used for bioprinting an organ defect on the human body surface or natural cavity organ defect, the base 31 can be bound or bonded and fixed on a surface of the human body near the organ defect, while the vertical tube 32 can be omitted or inserted into a natural lumen of the human body, and can be detachably connected to the first interface 18 of the implantable component 1 through the second interface 35.

As shown in FIG. 9C, a connectable component 3 of a device of in-situ 4D bioprinting organ of an embodiment of the present invention may comprise a base 31, a vertical tube 32A, a sealing valve 33A, a through hole 34A, a second interface 35A, a vertical tube 32B, a sealing valve 33B, a through hole 34B, and a second interface 35B. The base 31, the vertical tube 32A, the sealing valve 33A, the through hole 34A, and the second interface 35A can form a relatively independent structure of the connectable component 3, and the base 31, the vertical cylinder 32B, the sealing valve 33B, and the through hole 34B and the second interface 35B can form another relatively independent structure of the connectable component 3, configured to connect a relatively independent implantable component 1 and an external component 2, respectively, to achieve a structure or segmentation or organ reconstruction of two relatively independent organ defect areas by spray printing the bio ink. It should be noted that the connectable component 3 can include three or more sets of relatively independent connectable component 3 structures according to a requirement, configured to spray the bio ink to achieve multiple structures or multiple lobes or partitions or multiple organ reconstruction in the organ defect area.

As shown in FIG. 9D, a connectable component 3 of a device of in-situ 4D bioprinting organ according to the preferred embodiment of the present invention may comprise a base 31, a vertical tube 32, a sealing valve 33, a through-hole 34, and a second interface 35. The vertical tube 32 can be a soft tubular structure for inserting into a narrow and irregular areas or narrow natural channels in a deep part of the human body, such as a soft tubular structure where the vertical tube 32 can pass through a vagina, cervical canal and uterine body for printing a uterine defect.

As shown in FIGS. 2, 3, 10A to 10C, and 11, an external component 2 of a device of in-situ 4D bioprinting organ according to the preferred embodiment of the present invention can be detachably and fixedly connected to a base 31. The external component 2 may comprise a filling unit 21, a drainage unit 22, a drive unit 23, a control unit 24, a power 25. The filling unit 21, the drainage unit 22, the drive unit 23 and the control unit 24 can electrically connect to the power 25, and the control unit 24, the filling unit 21, the drainage unit 22, and the drive unit 23 can be electrically connected.

As shown in FIGS. 2, 3, 5A, 5B, and 10A, a filling unit 21 of a device of in-situ 4D bioprinting organ of an embodiment of the present invention may comprise a second connecting tube 211, a first injection pot 212, a storage bag 213, and a first micropump 214. A bio ink or artificial amniotic fluid can be injected into the storage bag 213 through the first injection pot 212. A control unit 24 can start the first micropump 214 according to an intelligent or manual control command, the bio ink or artificial amniotic fluid in the storage bag 213 can be sprayed to a residual wound of an organ defect area and generate organ tissue in layers through the second connecting tube 211, the first connecting tube 133, a traffic branch tube 134, a capillary net 13, and a plurality of second micropores 132. The artificial amniotic fluid can provide a suitable microenvironment for regeneration of the residual wound of the organ defect. It should be noted that the external component 2 of the device of in-situ 4D bioprinting organ can include a plurality of filling units 21 as needed, and the filling units can work in parallel to implement layered printing of the bio ink or injection of artificial amniotic fluid.

As shown in FIGS. 2, 3, 5A, 5B, and 10B, a filling unit 21 of a device of in-situ 4D bioprinting organ of an embodiment of the present invention may comprise a second connecting tube 211A, a second connecting tube 211B, a first injection pot 212, a storage bag 213, a first micropump 214, and a micro valve 215. A bio ink or artificial amniotic fluid can be injected into the storage bag 213 through the first injection pot 212. A control unit 24 can activate the first micropump 214 and the micro valve 215 according to an intelligent or manual control command. The bio ink or artificial amniotic fluid in the fluid storage bag 213 can enter two capillary pipelines composed of a first connecting tube 133, a traffic branch tube 134, a capillary net 13 and a plurality of second micropores 132 through the second connecting tube 211A and/or the second connecting tube 211B, and the bio ink can be layered and sprayed onto a residual wound of the two organ defect areas to generate organ tissue. The artificial amniotic fluid can provide a suitable microenvironment for the regeneration of organ tissue in the residual wound of organ defect area. It should be noted that a filling unit 21 of the device of in-situ 4D bioprinting organ can include three or more second connecting tubes 211 as needed. The bio ink can be layered and sprayed onto the residual wound of three or more organ defect areas to generate organ tissue.

As shown in FIGS. 2, 3, 5A, 5B, and 10C, a filling unit 21 of an in situ 4D bioprinting organ device according to the preferred embodiment of the present invention may comprise a second connecting tubes 211, a first injectable pot 212A, a liquid storage bag 213A, a first micropump 214A, a first injectable pot 212B, a liquid storage bag 213B, and a first micropump 214B. Different formulations of bio ink or artificial amniotic fluid can be inkjet into the liquid storage bag 213A and the liquid storage bag 213B through the first injectable pot 212A and the first injectable pot 212B, respectively. A control unit 24 can start the first micropump 214A and/or the first micropump 214B according to an intelligent or manual control command. The bio ink or artificial amniotic fluid inside the liquid storage bag 213A and/or the liquid storage bag 213B can enter a capillary pipeline composed of a first connecting tube 133, a traffic branch tube 134, a capillary net 13, and a plurality of second micropores 132 through the second connecting tube 211, and then the bio ink can be sprayed to a residual wound of two organ defect areas in layers and generate organ tissue. The injected artificial amniotic fluid provides a suitable microenvironment for the regeneration of organ tissue on the residual wound of organ defect areas.

As shown in FIGS. 2, 3 and 11, a drainage unit 22 of an in situ 4D bioprinting organ device according to an embodiment of the present invention may comprise a third connecting tube 221, a second micropump 225, a waste liquid bag 224, an exhaust hole 223, and a second injectable pot 222. The third connecting tube 221 can penetrate and open into a chamber 14, and a control unit 24 can start the second micropump 225 according to an intelligent or manual control command, then a liquid in the chamber 14 can enter the waste liquid bag 224 through the third connecting tube 221. A residual gas in the waste liquid bag 224 can be discharged through the exhaust hole 223. When the waste liquid bag 224 is filled with the liquid, the liquid can be extracted using a syringe through the second injecting pot 222. The liquid extracted can be used to analyze a secretion and metabolism status of organs in the chamber 14 through detection.

It should be noted that the control unit 24 may comprise a data storage processor and a wireless communication subunit, which are respectively used for real-time data processing and wireless data transmission.

The bio ink for printing can include at least one or more combinations of seed cells, collagen, cellulose, hyaluronic acid, and growth factors. The seed cells can include directed or pluripotent stem cells, mature organ cells, endothelial cells, and epithelial cells. The filled artificial amniotic fluid can be used to fill the chamber 14, so that the chamber 14 has a microenvironment similar to an amniotic cavity, and promotes the regeneration of organ defect area. Preferably, the artificial amniotic fluid can include additives, which can include one or more combinations of cytokines, growth factors, hormones, hemostatic agents, antibiotics, and enzymes.

As shown in FIGS. 2 to 4, a drive unit 23 of a device of in-situ 4D bioprinting organ of an embodiment of the present invention typically uses a micro motor as a driving source, preferably a micro stepper motor. A main body of the drive unit 23 can be fixed to a base 31, and the drive unit 23 can be detachably fixed connected to a capillary net 13 of an implantable component 1 through a gear rack 231. A control unit 24 can start the micro stepper motor according to an intelligent or manual control commands, and the capillary net 13 can be driven by the gear rack 231 to move back and forth along an inner wall 12 and an outer shell 11 in a chamber 14. As shown in FIG. 3, a drive unit 23 of a device of in-situ 4D bioprinting organ of an embodiment of the present invention can be configured with a plurality of subunits as needed, which can be detachably and fixedly connected to a capillary net 13A and capillary net 13B of an implantable component 1 through a gear rack 231A and a gear rack 231B, respectively. A control unit 24 can start a micro stepper motor according to an intelligent or manual control command, and the micro stepper motor can be activated through the gear rack 231A and the gear rack 231B drives the capillary net 13A and the capillary net 13B of the implantable component 1 to move back and forth along an inner wall 12A, an outer shell 11A, an inner wall 12B, and an outer shell 11B in a chamber 14A and chamber 14B, respectively.

It should be noted that a power 25 of a device of in-situ 4D bioprinting organ of an embodiment of the present invention can be provided by a rechargeable battery or an external power source.

An embodiment of the present invention provides a system for in-situ 4D bioprinting organs, which may comprise a device for in-situ 4D bioprinting organs, a bio ink, an artificial amniotic fluid, an IoT operating systems, a plurality of applications, and algorithms. The applications may comprise a mobile program and a service program. The algorithm can include a monitoring data fusion analysis, a bio ink inkjet scheme, an artificial amniotic fluid filling and drainage scheme, and a recognition of wound healing process.

An embodiment of the present invention provides a system for in-situ 4D bioprinting organ, which may comprise a 5G, a cloud server, a plurality of intelligent terminals, and a plurality of medical care units.

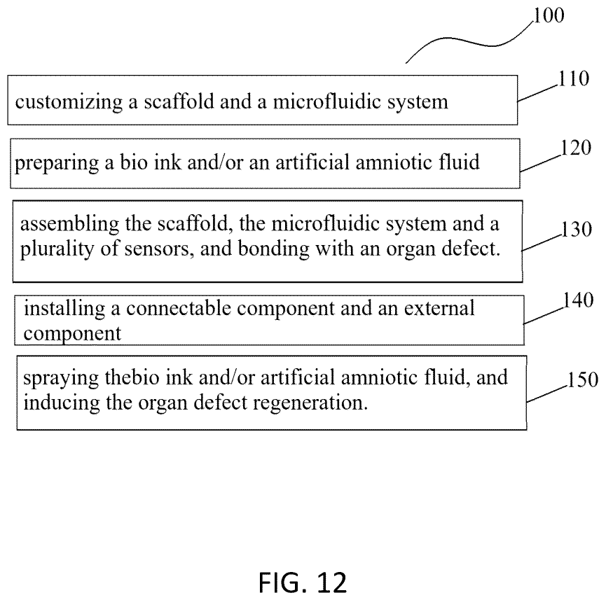

As shown in FIG. 12, an embodiment of the present invention provides a method process 100 for in-situ 4D bioprinting organ, which may comprise the following steps.

Step 110: Customize a scaffold for shaping an organ defect and a microfluidic system, the scaffold for shaping an organ defect including an inner wall, an outer shell, and a plurality of first micropores, wherein the microfluidic system includes a capillary net, a plurality of second micropores, and a first connecting tube.

The scaffold for shaping an organ defect and microfluidic system pipelines can be 3D modeled and 3D printed performed on organs in a chest, an abdominal cavity, or a natural lumen, such as a liver, kidneys, and uterus, based on organ data from a same age and/or body type group, as well as a shape and size of a residual organ. The scaffold for shaping an organ defect can be preferably made of biodegradable materials, and the microfluidic system pipeline can be made of biodegradable medical materials or non-biodegradable medical polymer materials.

The scaffold for shaping an organ defect and microfluidic system pipelines of a human body surface organ defect area, such as an outer ear, nose, trunk, and limb, can be modeled and printed in 3D using contralateral healthy organ data or referring to a previous photo data or human aesthetic standard. The scaffold for shaping an organ defect and microfluidic system pipelines of a human body surface organ defect can be made of biodegradable or non-degradable medical polymer materials, preferably transparent and non-degradable medical polymer materials, which is beneficial for reducing costs.

In addition, during a regeneration process of in-situ 4D bioprinting organ, the scaffold for shaping an organ defect and microfluidic system pipelines of the organ defect can be 3D modeled and 3D printed one or more times again according to an actual need.

Step 120: Prepare a bio ink and/or artificial amniotic fluid, the bio ink including seed cells, extracellular matrix, and growth factors.

The bio ink for in-situ 4D bioprinting organ defect may comprise a series of segmented bio inks, which can be configured to inkjet to form a plurality of different tissues. Taking in-situ 4D bioprinting liver defect as an example, the series of segmented bio inks can be configured to print a liver parenchyma and a liver outer wall. Taking in-situ 4D bioprinting nasal defect as an example, the series of segmented bio inks can be configured to print a nasal mucosal layer, a nasal cartilage and/or a cortical bone, a skin and a subcutaneous defect, with an intermittent or continuous printing in layers and zones. The segmented bio inks can include at least one or more combinations of seed cells, collagen, cellulose, hyaluronic acid, and growth factors, among which seed cells can include a plurality of directed and/or pluripotent stem cells, mature organ cells, endothelial cells, and epithelial cells.

The artificial amniotic fluid may comprise a series of segmented artificial amniotic fluids, which can be configured to create a simulated amniotic cavity microenvironment in the organ defect area to promote the organ regeneration. Preferably, the artificial amniotic fluids may comprise a plurality of additives, which can include one or more combinations of cytokines, growth factors, hormones, hemostatic agents, antibiotics, and enzymes.

Step 130: Assemble the scaffold for shaping an organ defect, a microfluidic system and a plurality of sensors, which being folded or compressed into a body through a minimally invasive incision or installed on the body surface, and connected and bonded with a residual of organ defect.

Before splicing and bonding the residual of organ defect, a pre-treatment is required, which can include making fresh wounds and improving hemostasis on the residual of organ defect.

When splicing and bonding the residual of organ defect, it is necessary to accurately align a medullary chamber of the organ stump with an inner wall of the scaffold for shaping an organ defect, and accurately align an outer shell of the organ stump with an outer shell of the scaffold for shaping an organ defect. The operation above mentioned can be performed under an endoscope or by a surgical robot.

Step 140: Install a connectable component and an external component.

As shown in FIGS. 2 and 3, a base 31 of a connectable component 3 can be generally set around a surgical incision, and a drive unit 23, a filling unit 21, a drainage unit 22, a control unit 24, and a power 25 of an external component 2 can be detachably fixed on the base 31. In addition, a power transmission subunit, a plurality of connecting tubes, circuits, and communication lines of the external component 2 can pass through a through hole 34 of the connectable component 3.

If a damaged organ being in-situ 4D bio-printed is a surface organ, such as an outer ear, a nose, a trunk, or a limb, the base 31 can be bound or pasted near the surface organ, a second interface 35 of the connectable component 3 can be disassembled to connect a first interface of the implantable component 1, and a power transmission subunit, a connecting tubes, circuits, and communication lines of an external component 2 can be connected to a microfluidic system pipelines, sensors, and temperature control subunit of the implantable component through a plurality of through holes of the connectable component 3.

Step 150: Spray the bio ink and filling the artificial amniotic fluid according to a procedure, and providing a local tissue regeneration microenvironment, thus inducing the tissue regeneration.

As shown in FIGS. 2, 3, 5A, 5B, 10C, and 11, a segmented bio ink or artificial amniotic fluid of a series of formulations can be inkjet into a storage bag 213 through a first injectable pot. A control unit 24 can start a first micropump 214 and/or micro valve according to an intelligent or manual control command, and the segmented bio ink or artificial amniotic fluid in the storage bag 213 can enter a capillary net 13, spray a layer of the segmented bio ink with a thickness about 1 mm or several mm to a fresh wound surface of the residual organ defect to generate organ tissue, inject the segmented artificial amniotic fluid into the chamber 14 to provide a suitable microenvironment for organ regeneration, and a temperature control unit 17 provides a temperature environment from 37.5° C. to 39.5° C. for organ regeneration. When an intelligent monitoring unit 16 observes that the segmented bio ink being sprayed has completed organ tissue regeneration, a control unit 24 can start a second micropump 225 according to an intelligent or manual control command, a liquid in the chamber 14 can enter a waste liquid bag 224, the control unit 24 can start the first micropump 214 and/or micro valve according to an intelligent or manual control command, and the segmented bio ink or artificial amniotic fluid in storage bag 213 can enter the capillary net 13, spray a layer of the segmented bio ink with a thickness about 1 mm or several mm to the fresh wound surface of the residual organ defect area to generate organ tissue, and then inject the segmented artificial amniotic fluid into the chamber 14 to provide a suitable microenvironment for organ regeneration until a repair and reconstruction of the organ defective is completed.

Step 160: Remove the connectable component and external component of the in-situ 4D bioprinting organ, removing the microfluidic system pipelines and sensors, and the scaffold for shaping an organ defect will degrade or be removed.

### Example 1: An In-Situ 4D Bioprinting for an External Ear Defect

A 3D scanning can be configured to collect data from a healthy external ear organ on a opposite side, then a 3D modeling can be followed by chirality rules, and then a 3D printing of a first stage scaffold for shaping the external ear defect and a microfluidic system pipeline can be performed. In addition, during a process of external ear regeneration, the 3D modeling and 3D printing of a second stage scaffold for shaping the external ear defect and a microfluidic system pipeline can be performed can be carried out according to an actual need.

A series of segmented bio ink and/or artificial amniotic fluid can be prepared, which can include seed cells, extracellular matrix, and growth factors that can differentiate into a skin, subcutaneous tissue, and ear cartilage.

A fresh wound in the external ear defect can be created, then the first stage scaffold for shaping the external ear defect and the microfluidic system pipeline can be assembled, then a plurality of sensors, temperature control unit, and a connectable component and external component can be installed. And then the segmented bio ink can be inkjet and printed in stages and layers according to a procedure, and the segmented artificial amniotic fluid can be filled to provide a local tissue regeneration microenvironment and induce the regeneration of the outer ear tissue. If necessary, the first stage scaffold for shaping the external ear defect and the microfluidic system pipeline can be replaced with the second stage scaffold for shaping the external ear defect and the microfluidic system pipeline. When a repair and reconstruction of the external ear defect is completed, the device of in-situ 4D bioprinting organ can be removed.

### Example 2: An In-Situ 4D Bioprinting for Reconstruction of Nasal Defects

A 3D scanning can be configured to collect data of a residual nasal structure, then a 3D modeling can be followed which combing with a patient recognized aesthetic standard, and then a 3D printing of a first stage scaffold for shaping the nasal defect and a microfluidic system pipeline can be performed. In addition, according to an actual need, the 3D modeling and 3D printing of a second stage scaffold for shaping the nasal defect and a microfluidic system pipeline can be carried out again.

A series of segmented bio ink and/or artificial amniotic fluid can be prepared, which can include seed cells, extracellular matrix, and growth factors that can differentiate into a skin, subcutaneous tissue, nasal mucosa, and nasal cartilage.

A fresh wound in the nasal defect can be created, then the first stage scaffold for shaping the nasal defect and the microfluidic system pipeline can be assembled, then a plurality of sensors, temperature control unit, and a connectable component and external component can be installed. And then the segmented bio ink can be inkjet and printed in stages and layers according to a procedure, and the segmented artificial amniotic fluid can be filled to provide a local tissue regeneration microenvironment and induce the regeneration of the nasal defect tissue. If necessary, the first stage scaffold for shaping the nasal defect and the microfluidic system pipeline can be replaced with the second stage scaffold for shaping the nasal defect and the microfluidic system pipeline. When a repair and reconstruction of the nasal defect is completed, the device of in-situ 4D bioprinting organ can be removed.

### Example 3: An In-Situ 4D Bioprinting for a Breast Reconstruction

If a breast conserving surgery is performed, a preoperative evaluation of breast cancer tissue resection volume, a 3D modeling and 3D printing of a scaffold for shaping a breast tissue defect and a microfluidic system pipeline can be carried out. The scaffold for shaping the breast tissue defect and the microfluidic system pipeline can be plant through a surgical incision, and a plurality of sensors and temperature control unit, a connectable component and an external component can be installed. A series of segmented bio ink and/or artificial amniotic fluid can be prepared, which can include seed cells that can differentiate into an adipose tissue and a nipple skin tissue, an extracellular matrix, and growth factors. In the breast defect area, the segmented bio ink can be inkjet and printed in layers and stages according to a procedure. The segmented artificial amniotic fluid can be filled to provide a local tissue regeneration microenvironment and induce the regeneration of adipose tissue and nipple skin tissue. When a repair and reconstruction of the breast defect, the device of in-situ 4D bioprinting organ can be removed.

If a routine radical mastectomy is performed, a preoperative 3D scanning can be configured to collect a breast data, a 3D modeling and 3D printing can be configured to preparation of a scaffold for shaping a breast tissue defect and a microfluidic system pipeline. After a complete or partial removal of the breast, the scaffold for shaping the breast tissue defect and the microfluidic system pipeline can be installed on a breast wound surface, a plurality of sensors, a temperature control unit, a connectable component and an external component can be installed. Then a prepared segmented bio and/or artificial amniotic fluid, which can include seed cells that differentiate into adipose tissue, skin tissue, subcutaneous tissue, extracellular matrix, and growth factors, can be sprayed and printed in layers and stage in the breast defect area according to a procedure to provide a local tissue regeneration microenvironment and induce the regeneration of adipose tissue and skin tissue. When a repair and reconstruction of the breast defect, the device of in-situ 4D bioprinting organ can be removed.

### Example 4: An In-Situ 4D Bioprinting for a Liver Defect

The in-situ 4D bioprinting for a liver defect is suitable for most liver resection or transplantation with insufficient tissue volume and can be performed simultaneously with the liver resection and/or transplantation surgery. A preoperative 3D modeling and 3D printing of a scaffold for shaping a liver defect and a microfluidic system pipeline can be carried out. After the liver resection and/or liver transplantation, the scaffold for shaping a liver defect and a microfluidic system pipeline can be installed in a wound surface of the liver defect area, at the same time, a plurality of sensors, a temperature control unit, a connectable component and an external component can be installed. Then a series of prepares segmented bio ink and/or artificial amniotic fluid, which can include seed cells that differentiate into liver cells and epithelial cells, extracellular matrix, and growth factors can be sprayed and printed in layers and stages in the liver defect area according to a procedure. When completing a repair and reconstruction of the liver defect, the device of in-situ 4D bioprinting organ can be removed.

### Example 5: An In-Situ 4D Bioprinting for a Kidney Defect

The in-situ 4D bioprinting for a kidney defect is suitable for most nephrectomies or transplantation with insufficient kidney tissue, and can be performed simultaneously with the nephrectomy or kidney transplant surgery. A preoperative 3D modeling and 3D printing of a scaffold for shaping a kidney defect and a microfluidic system pipeline can be carried out. After the nephrectomy and/or kidney transplantation, the scaffold for shaping a kidney defect and a microfluidic system pipeline can be installed in a wound surface of the kidney defect area, at the same time, a plurality of sensors, a temperature control unit, a connectable component and an external component can be installed. Then a series of prepares segmented bio ink and/or artificial amniotic fluid, which can include seed cells that differentiate into renal parenchymal cells, mucosal epithelial cells, extracellular matrix, and growth factors can be sprayed and printed in layers and stages in the kidney defect area according to a procedure. When completing a repair and reconstruction of the kidney defect, the device of in-situ 4D bioprinting organ can be removed.

### Example 6: An In-Situ 4D Bioprinting for a Brain Defect

The in-situ 4D bioprinting for a brain defect is suitable for most brain resection or large-scale old cerebral infarction and can be performed simultaneously with the brain resection surgery. A preoperative 3D modeling and 3D printing of a scaffold for shaping a brin defect and a microfluidic system pipeline can be carried out. The scaffold for shaping a brin defect and a microfluidic system pipeline can be installed in a wound surface of the brain defect area, at the same time, a plurality of sensors, a temperature control unit, a connectable component and an external component can be installed. Then a series of prepares segmented bio ink and/or artificial amniotic fluid, which can include seed cells that differentiate into neurons and glial cells, mucosal epithelial cells, extracellular matrix, and growth factors can be sprayed and printed in layers and stages in the brain defect area according to a procedure. When completing a repair and reconstruction of the brain defect, the device of in-situ 4D bioprinting organ can be removed.

### Example 7: An In-Situ 4D Bioprinting for a Trunk and/or Limb Defect

A 3D scanning can be used to collect data of trunk and/or limb defects, and/or an imaging can be used to evaluate an amount of necrotic tissue of trunk and/or limb. Then a 3D modeling and 3D printing of a scaffold for shaping the trunk and/or limb defects and a microfluidic system pipeline can be carried out. In addition, according to an actual need, the 3D modeling and 3D printing of a second stage of the scaffold for shaping the trunk and/or limb defects and the microfluidic system pipeline can be carried out again.

A series of segmented bio ink and/or artificial amniotic fluid can be prepared, which can include a plurality of seed cells, extracellular matrix, and growth factors, the seed cells can be configured to differentiate into a bone, a cartilage, a muscle, a fascia, a fat, a subcutaneous tissue, and a skin.

A fresh wound in the trunk and/or limb defects can be created, then the scaffold for shaping the trunk and/or limb defects and the microfluidic system pipeline can be assembled, then a plurality of sensors, temperature control unit, and a connectable component and external component can be installed. And then the segmented bio ink can be inkjet and printed in stages and layers according to a procedure, and the segmented artificial amniotic fluid can be filled to provide a local tissue regeneration microenvironment and induce the regeneration of the trunk and/or limb defects tissue. If necessary, the scaffold for shaping the trunk and/or limb defects and the microfluidic system pipeline can be replaced with the second stage scaffold for shaping the trunk and/or limb defects and the microfluidic system pipeline. When a repair and reconstruction of the trunk and/or limb defects is completed, the device of in-situ 4D bioprinting organ can be removed.

### Example 8: An In-Situ 4D Bioprinting for a Heart Defect

The in-situ 4D bioprinting for a heart defect is suitable for a partial cardiac resection and/or a local large-scale infarction. A preoperative 3D modeling and 3D printing of a scaffold for shaping a heart defect and a microfluidic system pipeline can be carried out. The scaffold for shaping the heart defect and the microfluidic system pipeline can be installed in a wound surface of the heart defect area, at the same time, a plurality of sensors, a temperature control unit, a connectable component and an external component can be installed. Then a series of prepares segmented bio ink and/or artificial amniotic fluid, which can include seed cells that differentiate into myocardial cells and vascular endothelial cells, extracellular matrix, and growth factors can be sprayed and printed in layers and stages in the heart defect area according to a procedure, providing a local tissue regeneration microenvironment and inducing heart tissue, ventricle, atrium, and valve regeneration. When completing a repair and reconstruction of the heart defect, the device of in-situ 4D bioprinting organ can be removed.

### Example 9: An In-Situ 4D Bioprinting for a Genitalia Defect

The in-situ 4D bioprinting for a genitalia defect is suitable for a partial genital resection or reconstruction. A preoperative 3D modeling and 3D printing of a scaffold for shaping a genitalia defect and a microfluidic system pipeline can be carried out. The scaffold for shaping the genitalia defect and the microfluidic system pipeline can be installed in a wound surface of the genitalia defect area, at the same time, a plurality of sensors, a temperature control unit, a connectable component and an external component can be installed. Then a series of prepares segmented bio ink and/or artificial amniotic fluid, which can include seed cells that differentiate into genital cells and vascular endothelial cells, extracellular matrix, and growth factors can be sprayed and printed in layers and stages in the heart defect area according to a procedure, providing a local tissue regeneration microenvironment and inducing genitalia tissue regeneration. When completing a repair and reconstruction of the genitalia defect, the device of in-situ 4D bioprinting organ can be removed.

### Example 10: An In-Situ 4D Bioprinting for a Tongue Defect

The in-situ 4D bioprinting for a tongue defect is suitable for a partial tongue resection. A preoperative 3D modeling and 3D printing of a scaffold for shaping a tongue defect and a microfluidic system pipeline can be carried out. The scaffold for shaping the tongue defect and the microfluidic system pipeline can be installed in a wound surface of the tongue defect area, at the same time, a plurality of sensors, a temperature control unit, a connectable component and an external component can be installed. Then a series of prepares segmented bio ink and/or artificial amniotic fluid, which can include seed cells that differentiate into tongue cells and mucosal endothelial cells, extracellular matrix, and growth factors can be sprayed and printed in layers and stages in the tongue defect area according to a procedure, providing a local tissue regeneration microenvironment and inducing tongue tissue regeneration. When completing a repair and reconstruction of the tongue defect, the device of in-situ 4D bioprinting organ can be removed.

### Example 11: An In-Situ 4D Bioprinting for a Lung Defect

The in-situ 4D bioprinting for a tongue defect is suitable for a partial lung resection. A preoperative 3D modeling and 3D printing of a scaffold for shaping a lung defect and a microfluidic system pipeline can be carried out. The scaffold for shaping the lung defect and the microfluidic system pipeline can be installed in a wound surface of the lung defect area, at the same time, a plurality of sensors, a temperature control unit, a connectable component and an external component can be installed. Then a series of prepared segmented bio ink and/or artificial amniotic fluid, which can include seed cells that differentiate into epithelial cells and mucosal endothelial cells, extracellular matrix, and growth factors can be sprayed and printed in layers and stages in the lung defect area according to a procedure, providing a local tissue regeneration microenvironment and inducing the lung tissue regeneration. When completing a repair and reconstruction of the lung defect, the device of in-situ 4D bioprinting organ can be removed.

### Example 12: An In-Situ 4D Bioprinting for a Uterine Defect

The in-situ 4D bioprinting for a uterine defect is suitable for a partial hysterectomy. A preoperative 3D modeling and 3D printing of a scaffold for shaping a uterine defect and a microfluidic system pipeline can be carried out. The scaffold for shaping the uterine defect and the microfluidic system pipeline can be installed in a wound surface of the uterine defect area, at the same time, a plurality of sensors, a temperature control unit, a connectable component and an external component can be installed. Then a series of prepares segmented bio ink and/or artificial amniotic fluid, which can include seed cells that differentiate into smooth muscle cells, epithelial cells and endothelial cells, extracellular matrix, and growth factors can be sprayed and printed in layers and stages in the uterine defect area according to a procedure, providing a local tissue regeneration microenvironment and inducing the uterine tissue regeneration. When completing a repair and reconstruction of the uterine defect, the device of in-situ 4D bioprinting organ can be removed.

### Example 13: An In-Situ 4D Bioprinting for a Craniofacial Defect

A 3D scanning can be used to collect a structural data of a residual craniofacial structure. Then a 3D modeling and 3D printing of a first stage scaffold for shaping a craniofacial defect and a microfluidic system pipeline can be carried out combine with an aesthetic standard recognized by a patient. In addition, according to an actual need, the 3D modeling and 3D printing of a second stage scaffold for shaping the craniofacial defect and the microfluidic system pipeline can be carried out again. And then a series of segmented bio inks and/or artificial amniotic fluids can be prepared. The bio inks may comprise a plurality of seed cells, extracellular matrix and growth factors. The seed cells can be configured to differentiated into a bone, a cartilage, a muscle, a nerve, a fat, a skin, a subcutaneous tissue and a mucosa.

A fresh wound in the craniofacial defect can be created, then the scaffold for shaping the craniofacial defect and the microfluidic system pipeline can be assembled, then a plurality of sensors, temperature control unit, and a connectable component and external component can be installed. And then the segmented bio inks can be inkjet and printed in stages and layers according to a procedure, and the segmented artificial amniotic fluid can be filled to provide a local tissue regeneration microenvironment and induce the regeneration of the craniofacial defect tissue. If necessary, the scaffold for shaping the craniofacial defect and the microfluidic system pipeline can be replaced with the second stage scaffold for shaping the craniofacial defect and the microfluidic system pipeline. When a repair and reconstruction of the craniofacial defect is completed, the device of in-situ 4D bioprinting organ can be removed.

### Example 14: An In-Situ 4D Bioprinting for a Finger and/or Toe Defect

A 3D scanning can be used to collect a structural data of a residual finger and/or toe structure. Then a 3D modeling and 3D printing of a first stage scaffold for shaping a finger and/or toe defect and a microfluidic system pipeline can be carried out combine with a normal finger and/or toe imaging data. In addition, according to an actual need, the 3D modeling and 3D printing of a second stage scaffold for shaping the finger and/or toe defect and the microfluidic system pipeline can be carried out again. And then a series of segmented bio inks and/or artificial amniotic fluids can be prepared. The bio inks may comprise a plurality of seed cells, extracellular matrix and growth factors. The seed cells can be configured to differentiated into a bone, a cartilage, a joint, a muscle, a fascia, a tendon, a nerve, a fat, a subcutaneous tissue and a skin.

A fresh wound in the finger and/or toe defect can be created, then the scaffold for shaping the finger and/or toe defect and the microfluidic system pipeline can be assembled, then a plurality of sensors, temperature control unit, and a connectable component and external component can be installed. And then the segmented bio inks can be inkjet and printed in stages and layers according to a procedure, and the segmented artificial amniotic fluid can be filled to provide a local tissue regeneration microenvironment and induce the regeneration of the finger and/or toe defect tissue. If necessary, the scaffold for shaping the finger and/or toe defect and the microfluidic system pipeline can be replaced with the second stage scaffold for shaping the finger and/or toe defect and the microfluidic system pipeline. When a repair and reconstruction of the finger and/or toe defect is completed, the device of in-situ 4D bioprinting organ can be removed.

### Example 15: An In-Situ 4D Bioprinting for an Anus Defect

A 3D modeling and 3D printing of a first stage scaffold for shaping an anus defect and a microfluidic system pipeline can be carried out. In addition, according to an actual need, the 3D modeling and 3D printing of a second stage scaffold for shaping the anus defect and the microfluidic system pipeline can be carried out again. And then a series of segmented bio inks and/or artificial amniotic fluids can be prepared. The bio inks may comprise a plurality of seed cells, extracellular matrix and growth factors. The seed cells can be configured to differentiated into a muscle, a fascia, a tendon, a nerve, a fat, a mucosal tissue, and a skin.

A fresh wound in the anus defect can be created, then the scaffold for shaping the anus defect and the microfluidic system pipeline can be assembled, then a plurality of sensors, temperature control unit, and a connectable component and external component can be installed. And then the segmented bio inks can be inkjet and printed in stages and layers according to a procedure, and the segmented artificial amniotic fluid can be filled to provide a local tissue regeneration microenvironment and induce the regeneration of the anus defect tissue. If necessary, the scaffold for shaping the anus defect and the microfluidic system pipeline can be replaced with the second stage scaffold for shaping the anus defect and the microfluidic system pipeline. When a repair and reconstruction of the anus defect is completed, the device of in-situ 4D bioprinting organ can be removed.

### Example 16: An In-Situ 4D Bioprinting for a Gastrointestinal Tract Defect

An implementation step of the in-situ 4D bioprinting for a gastrointestinal tract defect can be referred to Example 15.

### Example 17: An In-Situ 4D Bioprinting for a Pancreatic Defect

A 3D modeling and 3D printing of a first stage scaffold for shaping a pancreatic defect and a microfluidic system pipeline can be carried out. In addition, according to an actual need, the 3D modeling and 3D printing of a second stage scaffold for shaping the pancreatic defect and the microfluidic system pipeline can be carried out again. And then a series of segmented bio inks and/or artificial amniotic fluids can be prepared. The bio inks may comprise a plurality of seed cells, extracellular matrix and growth factors. The seed cells can be configured to differentiated into a A cell, a B cell, a Delta cell, a pancreatic polypeptide cell, and a mucosal tissue muscle.

A fresh wound in the anus defect can be created, then the scaffold for shaping the pancreatic defect and the microfluidic system pipeline can be assembled, then a plurality of sensors, temperature control unit, and a connectable component and external component can be installed. And then the segmented bio inks can be inkjet and printed in stages and layers according to a procedure, and the segmented artificial amniotic fluid can be filled to provide a local tissue regeneration microenvironment and induce the regeneration of the pancreatic defect tissue. If necessary, the scaffold for shaping the pancreatic defect and the microfluidic system pipeline can be replaced with the second stage scaffold for shaping the pancreatic defect and the microfluidic system pipeline. When a repair and reconstruction of the pancreatic defect is completed, the device of in-situ 4D bioprinting organ can be removed.

### Example 18: An In-Situ 4D Bioprinting for a Spleen Defect

An implementation step of an in-situ 4D bioprinting for a spleen defect can be referred to Example 17.

The above is only the embodiments of the present invention and does not impose any limitations on the technical scope of the present invention. Therefore, any minor modifications, equivalent changes, or modifications made to the above embodiments based on the technical essence of the present invention still fall within the scope of the technical solution of the present invention. Professionals should be aware that they can use different methods to achieve the described functions for each specific application, but such implementation should not be considered beyond the scope of the present invention.

What is claimed is:

1. A device of in-situ 4D bioprinting organ for repairing an organ defect, comprising:
an implantable component configured to connect an organ defect and spray a bio ink to repair the organ defect;
an external component configured to provide a support to the implantable component; and
a connectable component configured to connect the implantable component and the external component.

2. The device, as recited in claim 1, wherein the implantable component comprises:
a scaffold configured to shape the organ defect, which comprises an inner wall, an outer shell and a plurality of first micropores,
a microfluidic system configured to inkjet the bio ink, which comprises a capillary net, a plurality of second micropores and a first connecting tube, and
a monitoring unit configured to collect data from the organ defect area, which comprises a plurality of sensors.

3. The device, as recited in claim 2, wherein the microfluidic system comprises a plurality of microfluidic pipelines configured to inkjet the bio ink to realize a regeneration of one of a plurality of partitions of the organ defect and a plurality of organ defects.

4. The device, as recited in claim 1, wherein the external component comprises:
a filling unit configured to inkjet at least one of the bio ink and an artificial amniotic fluid into a microfluidic system,
a drainage unit configured to discharge waste liquid from an organ defect area,
an intelligent motor configured to drive the artificial amniotic fluid to move, and
a control unit configured to control the filling unit, the drainage unit and the intelligent motor.

5. The device, as recited in claim 4, wherein the bio ink is a segmented bio ink, which comprises one or more combinations of a seed cell, a collagen, a cellulose, a hyaluronic acid, and a growth factor.

6. The device, as recited in claim 1, wherein the connectable component comprises a base configured to anchor the implantable component and the external component, which includes an abutment and a through hole.

7. A system of in-situ 4D bioprinting organ for completing in-situ bioprinting organ defect, comprising:
a device of in-situ 4D bioprinting organ configured to implement in-situ bioprinting organ defect;
a bio ink and an artificial amniotic fluid configured to provide bioprinting materials and local microenvironment for the device of in-situ 4D bioprinting organ;
an algorithm configured to analyze data, formulate a printing plan, and recognize a wound; and
an application program configured to run the algorithm.

8. A method for in-situ 4D bioprinting organ, including:
customizing a scaffold and a microfluidic system, wherein the scaffold includes an inner wall, an outer shell and a plurality of first micropores, and the microfluidic system includes a capillary net, a plurality of second micropores and a first connecting tube;
preparing one of a bio ink and an artificial amniotic fluid,
assembling the scaffold, a microfluidic system and a plurality of sensors, and bonding with a residual of an organ defect;
installing a connectable component and an external component; and
spraying the bio ink, filling the artificial amniotic fluid according to a procedure, and implementing bioprinting the organ defect.

9. The method, as recited in claim 8, further comprising:
real-time collecting a wound microcirculation data, a tissue regeneration progress data, a temperature data, and a pH data.

10. The method, as recited in claim 8, further comprising:
removing the connectable component, the external component and the microfluidic system.

11. The method, as recited in claim 8, further comprising:
customizing the scaffold for shaping an external ear defect and the microfluidic system configured for bioprinting the external ear.

12. The method, as recited in claim 11, further comprising:
customizing the scaffold for shaping a nasal defect and the microfluidic system configured for bioprinting the nasal defect.

13. The method, as recited in claim 12, further comprising:
customizing the scaffold for shaping a breast defect and the microfluidic system configured for bioprinting the breast defect.

14. The method, as recited in claim 13, further comprising:
customizing the scaffold for shaping a liver defect and the microfluidic system configured for bioprinting the liver defect.

15. The method, as recited in claim 14, further comprising:
customizing the scaffold for shaping a kidney defect and the microfluidic system configured for bioprinting the kidney defect.

16. The method, as recited in claim 15, further comprising:
customizing the scaffold for shaping a brain defect and the microfluidic system configured for bioprinting the brain defect.

17. The method, as recited in claim 16, further comprising:
customizing the scaffold for shaping a trunk and/or limb defect and the microfluidic system configured for bioprinting the trunk and/or limb defect.

18. The method, as recited in claim 17, further comprising:
customizing the scaffold for shaping a heart defect and the microfluidic system configured for bioprinting the heart defect.

19. The method, as recited in claim 18, further comprising:
customizing the scaffold for shaping a genital defect and the microfluidic system configured for bioprinting the genital defect.

20. The method, as recited in claim 19, further comprising:
customizing the scaffold for shaping a tongue defect and the microfluidic system configured for bioprinting the tongue defect.
